SEQUENCE LISTING (2 sequences)

SEQ ID NO: 1 - protein, 54 aa, Artificial Sequence
  Synthetic protein
MRGSHHHHHHGSIEGRVKEITFLKNTAPQMLRELQETNAALQDVRELLRQQSKL

SEQ ID NO: 2 - protein, 44 aa, Artificial Sequence
  Synthetic protein
GSIEGRVKEITFLKNTAPQMLRELQETNAALQDVRELLRQQSKL

(19) United States
(12) Patent Application Publication
(10) Pub. No.: US 2022/0096709 A1
(43) Pub. Date: Mar. 31, 2022

(54) WOUND HEALING THERAPEUTIC HYDROGELS

(71) Applicant: New York University, New York, NY (US)

(72) Inventors: Piul Rabbani, Brooklyn, NY (US); Joseph Kuhn, Brooklyn, NY (US); Jin Kim Montclare, New York, NY (US); Priya Katyal, Mineola, NY (US); Michael Meleties, Whitestone, NY (US); Juan Francisco Cortes Troncoso, Santiago (CL)

(21) Appl. No.: 17/490,156

(22) Filed: Sep. 30, 2021

Related U.S. Application Data
(60) Provisional application No. 63/085,541, filed on Sep. 30, 2020.

(51) Int. Cl. A61L 26/00; A61K 31/4164; C12N 5/077

(52) U.S. Cl. CPC A61L 26/0047; A61K 31/4164; B82Y 40/00; A61L 26/008; C12N 5/0669; A61L 26/0066

(57) ABSTRACT

Provided are hydrogel-based compositions and materials for wound healing and methods of using same. The hydrogel comprises nanofibers formed from protein Q, which is a variant of the cartilage oligomeric matrix protein coiled coil (COMPcc) protein, or a protein having at least 85% homology with protein Q. The hydrogel has one or more wound healing agents distributed therein and associated with the Q fibers. The wound healing agent may be exosomes, which may be exosomes produced by cells, such as exosomes produced by multipotent stromal cells and/or one or more triterpenoids. The hydrogels may be used in treatment of wounds, such as chronic wounds.

### CROSS-REFERENCE TO RELATED APPLICATIONS

This application claims priority to U.S. Provisional application No. 63/085,541, filed on Sep. 30, 2020, the disclosure of which is incorporated herein by reference.

### STATEMENT REGARDING FEDERALLY SPONSORED RESEARCH

This invention was made with government support under grant no. 1840984 awarded by the National Science Foundation. The government has certain rights in the invention.

### BACKGROUND OF THE INVENTION

Chronic wounds are typically those that do not heal in about 30 days. Standards of care for such wounds involve addressing patient factors, such as nutritional and hydration status, mobility, infection, chronic disease, medications as well as social history. Medical care involves physical or biochemical debridement, weight off-loading, antibiotics and frequent dressings. Chronic disease like diabetes can be managed through glycemic control and lifestyle changes. However, these interventions do not prevent healing complications secondary to diabetes.

The impact on health care costs due to such chronic wounds is significant. Diabetic wounds (including ulcers) pose a $28 billion socio-economic dilemma in the United States alone (Sen 2019, Adv Wound Care (New Rochelle), Feb 1;8(2)39-48). The Global Lower Extremity Amputation Study Group found that 14-24% of patients with foot ulcers will sustain an amputation, a complication that drastically inflates health care expenditures and reduces quality of life (Jeffcoate 2006, Diabetes Care, Aug; 29(8):1784-7; Lipsky 2011, Diabetes Care, 2011 Aug;34(8):1695-700). The likelihood of amputation in people with diabetes is at least 10-20 times that of patients without diabetes (IDF 2019 report, diabetesatlas.org/en/resources/). Limb/digit amputations occur every 30 seconds around the world and are a direct morbid consequence of chronic non-healing wounds.

Currently, there are no therapies approved for efficacy, other than Regranex, which was FDA approved in 1997. Regranex is not widely used due to its cost and side effect profile with extended use (Papanas 2010, Drug Saf. 2010 Jun 1;33(6)455-61; Wieman 1998, Diabetes Care, 1998 May;21(5):822-7). Approved biologics for wound healing have not met efficacy endpoints, only safety ones.

### SUMMARY OF THE DISCLOSURE

The present disclosure provides hydrogel-based compositions and materials for wound healing. The hydrogel comprises nanofibers comprising protein Q, which is a variant of the coiled-coil region of cartilage oligomeric matrix protein (COMPcc) protein. The hydrogel comprising nanofibers comprising protein Q has distributed therein and associated with the Q fibers, one or more wound healing agents. The wound healing agent may be exosomes, which may be exosomes produced by cells, such as exosomes produced by multipotent stromal cells (e.g., such as those obtained from human bone marrow), or may be one or more triterpenoids, or a combination of exosomes and one or more triterpenoids.

The disclosure also provides methods for wound healing comprising contacting the wound with the compositions described herein. In an aspect, this disclosure provides a method of treatment of wounds by applying to the wound or near the wound a hydrogel composition comprising fibers of Q protein (SEQ ID NO:1 or 2, or comprising some molecules of SEQ ID NO:1 and some SEQ ID NO:2), having distributed therein one or more wound healing therapeutic agents. In embodiments, the wound healing therapeutic agents may be exosomes (e.g., isolated from cultures of multipotent stromal cells, which may be human stromal cells) and/or triterpenoids. The wounds may be chronic wounds, such as diabetic wounds and chronic ulcers.

### BRIEF DESCRIPTION OF THE FIGURES

For a fuller understanding of the nature and objects of the disclosure, reference should be made to the following detailed description taken in conjunction with the accompanying figures.

FIG. 1. Characterization of BM-MSCs and confirmation of stromal cell identity from hypoxic culture. Flow cytometric analysis of cell surface identifying proteins on BM-MSCs from healthy human donors. (A) Typical forward vs secondary scatter of adult BM-MSCs in monolayer cultures. The living cells were selected and analyzed further. BM-MSCs are not hematopoietic or monocytic in origin, as seen from lack of expression of indicated markers (CD45, CD34, CD19, CD11b, MHC-II) and coincidence with the isotype control peak in the histogram. (B) BM-MSCs, cultured in hypoxia demonstrate internationally accepted cell surface protein expression criteria for identification of multipotent stromal cells, CD44, CD106, CD90, CD73, CD105. The BM-MSCs have very low expression of CD36, which is associated with adipose tissue-derived MSCs instead.

FIG. 2. BM-MSCs, cultured in 5% O₂, retain multi-lineage differentiation capacity, as demonstrated by differentiation along chondrogenic, osteogenic and adipogenic lineages. These are the typical lineages a multipotent cell of stromal or mesenchymal origin should have capacity to terminally differentiate into. Chondrogenic differentiation is detected using Safranin-o staining, osteogenic differentiation is detected using Alizarin Red staining and adipogenic differentiation is detected using Oil Red O staining.

FIG. 3. MSC priming conditions affect exosome contents and pathways affected. RNASeq analysis of hypoxic exosome cargo vs normoxic, with>2 fold change over normoxic. Line denotes statistically significant gene ontology terms that have p<0.05. Arrows, genes enriching for critical pathways important in wound healing.

FIG. 4. Characterization of exosomes isolated from human bone marrow-derived multipotent stromal cells, under defined cell culture conditions described herein (A, B, C). (A) Immunoblot of BM-MSC derived exosomes showing detection of CD9, CD81 and TSG101, all associated with exosomes. The exosome samples do not express calnexin or GM-130 which are associated with larger apoptotic bodies and Golgi. The 2^{nd} column in the immunoblot contains conditioned medium from the BM-MSC cell cultures, but prior to concentration and isolation of exosomes. Hence, even if exosomes are present, their associated proteins are too dilute and below the detection limit of this assay. The schematic depicts typical locations of the exosome-associated proteins. (B) Nanoparticle tracking analysis conveys the diameter range and concentration of exosomes, once isolated from cell culture media. Shaded zones indicate range for standard deviations. (C) TEM, at 2 different magnifications of exosome preps from BM-MSCs.

FIG. 5. Inter-donor variability in total RNA carried by MSC exosomes is minimal. (A) and (B) are intensity heat maps showing gene expression in MSCs (A) and corresponding exosomes (B) from at least 3 different individuals. System Biosciences performed next generation RNA sequencing on the exosomes, while NYU's Genome Technology Center performed the next generation sequencing of the BM-MSCs. The scale on each shows expression level of RNAs detected. The similar color patterns indicate that despite genetic variation among human beings, BM-MSCs respond similarly to our culture methods to generate reproducible RNAs. Our subsequent exosome isolation protocols also enrich for exosomes that carry similar RNAs as well.

FIG. 6. No gelation is observed when a small molecule is added to the Q solution prior to incubating at 4° C. (A). Schematic showing that the addition of exosomes prior to incubation did not hinder with the network formation with gelation observed within 4 days at 4° C. (B).

FIG. 7. Micrographs showing network formation of (A) Q gel diluted to a concentration of 50 μM and B) in the presence of exosomes. (B) is a TEM showing exosomes are interspersed with the fibers and are indicated by the arrows. The scale bar in panel (A) is 200 nm while in panel (B) is 1 μm.

FIG. 8. Time to wound closure of 10 mm diameter full-thickness or excisional wounds. WT, wild type non-diabetic mice. Lepr^{db/db} (Db/db), type II diabetic mice with severe wound healing delay. BM-MSC Exo, human bone marrow-derived multipotent stromal cell exosomes. Administration of human BM-MSC exosomes locally on type 2 diabetic db/db wounds significantly accelerates wound closure, compared to the untreated wound, and within range of the WT wound closure time. *, p<0.05.

FIG. 9. Characterization of Exo-Q gel. Exo-Q gel prior to application on wounds.

FIG. 10A Photographs of diabetic wound area over time, comparing Exo-Q-treated wounds against vehicle-treated ones. FIG. 10A also shows 2 doses of Exo-Q treatment and times to closure. FIGS. 10B and 10C show areas under the curves for the plots from (10B). **, p<0.01; ns, not significant.

FIG. 11. Rheological properties of CDDO-Im bound Q hydrogel compared to Q hydrogel (Nrf2-Q). The storage moduli (G′) is higher compared to loss moduli (G″) at 4° C. under 5% oscillatory strain, confirming the elastic nature of both hydrogels.

FIG. 12. Wound healing dynamics with Nrf2-Q treatment. (A) Photographs of 6 mm diameter diabetic wounds over time with Nrf2-Q, Q (vehicle) or no treatment. (B) Wound area plotted over time. (C) Time to complete wound closure per treatment. (D) Wound burden (area under curve) of varying treatments in (B). *, p<0.05, ***, p<0.001, ns, not significant.

FIG. 13. Gene expression analysis in Nrf2-Q-treated wounds. A) Antioxidant transcripts, downstream of Nrf2. *, p<0.05, compared to Q alone. B) Growth factor transcripts induced by Nrf2-Q topical treatment. *, p<0.05. All data points, n=3.

FIG. 14. Histomicrograph representing wound histology after treatment with Exo-Q (3×10⁹ dose). Immunostaining was done with CD31, which detects endothelial cells, coupled with hematoxylin staining for nuclei. Dashed lines indicate area of granulation tissue, which is expanded following treatment with Exo-Q compared to the vehicle Q only.

FIG. 15. Gene expression analysis of growth factors of Exo-Q treated wounds. *, p<0.05, **, p<0.01, n=4.

FIG. 16. Thermoresponsiveness of Exo-Q hydrogels. Exo-Q gels stored at 4° C. are applied onto dorsal wounds on mice and photographed to capture the phase shift over time, from viscous to less viscous gel upon contact with the warmer mouse wound skin. The hydrogel can be followed above the silicone stent.

FIG. 17. 10 mm diameter full-thickness/excisional wounds on Lepr db/db (type 2 diabetic mouse) skin were treated topically by applying a liquid suspension of exosomes to the wound at post-operative day 1. This administration route did not affect the wound healing or closure time of the diabetic wounds. The time to closure with the topical liquid suspension were similar to untreated diabetic wounds and significantly higher than that of wild type non-diabetic wounds

### DESCRIPTION OF THE DISCLOSURE

This disclosure provides compositions useful for wound healing. The compositions can be used as via any cutaneous route (e.g., including administration via needle or microneedle) such as via local, topical, and/or transdermal applications. The compositions comprise hydrogel-based materials comprising wound healing agents. The hydrogel comprises fibers made of a variant (Q) of the coiled coil region of cartilage oligomeric matrix protein (COMPcc). The hydrogel comprises a network of fibers of the Q protein, and dispersed in the hydrogel and associated with the fibers are present wound healing agents. When small molecules are used as wound healing agents (such as triterpenoids), which may be hydrophobic, it is considered that they interact with the hydrophobic core of Q and also stabilize the hydrogels via other non-covalent interactions, as indicated by increase in elastic modulus. Exosomes are extracellular vesicles that pack together lipids, RNA, proteins etc. These vesicles may be non-covalently bound to Q.

The term “treatment” as used herein refers to reduction or delay in one or more symptoms or features associated with the presence of the particular condition being treated. Treatment does not necessarily mean complete cure and does not preclude relapse. Treatment may be carried out over a short period of time (days, weeks), or over a long period of time (months) or may be on a continuous basis (e.g., in the form of a maintenance therapy). Treatment may be continual or intermittent.

The term “therapeutically effective” dose or amount as used herein is the amount sufficient to achieve, in a single or multiple doses, the intended purpose of treatment. For example, an effective amount for effect wound healing is the amount sufficient to achieve one or more indicators of wound healing. The exact amount desired or required will vary depending on the mode of administration, patient specifics and the like. Appropriate effective amounts or the length of treatment can be determined by one of ordinary skill in the art (such as a clinician) with the benefit of the present disclosure.

The term “chronic wound” as used herein means a wound that does not heal within 30 days. Chronic wounds may further be defined as ones that do not progress at a measurable, sustained rate, indicative of a failure to progress through a typical tissue repair and regeneration sequence. For example, the percentage reduction or change in wound area in 4 weeks can indicate chronic nature or a non-acute wound. If the wounds do not have ˜50% reduction in area within 30 days, it may be considered chronic.

Where a range of values is provided in this disclosure, it should be understood that each intervening value, to the tenth of the unit of the lower limit between the upper and lower limit of that range, and any other intervening value in that stated range is encompassed within the disclosure, unless clearly indicated otherwise. The upper and lower limits of these smaller ranges may independently be included in the smaller ranges encompassed within the disclosure.

As used in this disclosure, the singular forms include the plural forms and vice versa unless the context clearly indicates otherwise.

In some embodiments, the disclosure broadly encompasses normalizing oxidative burden in diabetic wound beds by using engineered protein for effective delivery and release of materials.

In an aspect, this disclosure provides compositions for wound healing comprising wound healing agents distributed in a hydrogel comprising a variant of a coiled-coil domain of cartilage oligomeric matrix protein (COMPcc). The COMPcc variant may be referred to herein as Q. In an embodiment, the wound healing agent is exosomes isolated from multipotent stromal cells. In an embodiment, the wound healing agent is triterpenoids. The wound healing agents may be uniformly distributed in the hydrogel. A hydrogel may be in various forms, such as a film, a sheet, a monolith. and the like.

When the wound healing agent is an exosome, the present hydrogel material comprising the exosomes may be referred to herein as Exo-Q. When the wound healing agent is a triterpenoid or plurality of triterpenoids, the present hydrogel material comprising the triterpenoids may be referred to herein as Nrf2-Q. The triterpenoids may be semi-synthetic oleanane triterpenoids.

The hydrogels used in the present disclosure are based on a coiled-coil protein Q, which is an engineered variant of the coiled-coil domain of cartilage oligomeric matrix protein (COMPcc) (U.S. Pat. No. 9,777,041, the relevant portions of which are incorporated herein by reference). The sequence of Q is as follows:

(SEQ ID NO: 1)
MRGSHHHHHHGSIEGRVKEITFLKNTAPQMLRELQETNAALQDVRELLRQ
QSKL.

The sequence used for Q may also be without the poly his tag and amino acids from sequencing. Hence the sequence of Q may be: GSIEGRVKEITFLKNTAPQMLRELQETNAALQDVRELLRQQSKL (SEQ ID NO:2) or a variant that may have at least 85% identity with the sequence of SEQ ID NO:1 or SEQ ID NO:2 with substantially the same properties as Q of SEQ ID NO:1 or 2.

Q protein can be purified under native conditions using 50 mM tris hydrochloride, pH at least 8 (e.g., 8 to 10), and 500 mM NaCl. At a concentration of 2 mM, Q protein self-assembles to form a hydrogel at 4° C. Q demonstrates a UCST (upper critical solution temperature) type behavior, exhibiting gel-sol transition above its UCST (-16° C.) (Hill 2019, Biomacromolecules 2019, 20, 9, 3340-3351).

Q may also be purified as follows under denaturing conditions (50 mM Tris-HCl, 0.5 M NaCl, 20 mM imidazole, 6 M urea, pH 8 buffer). Here, urea is the denaturing agent. Purification in the absence of any chaotropic agent (e.g., urea) is referred as native conditions. Q self-assembles to form a hydrogel under native conditions.

Q may also be purified as follows. Q protein can be expressed in chemically competent M15MA Escherichia coli cells carrying the kanamycin-resistant pREP4 plasmid and induced with isopropyl B-D-1-thiogalactopyranoside (IPTG) at a final concentration of (200 μg mL⁻¹). The cells can be harvested and stored at −80° C. until purification. His-tag bearing Q protein can be purified using buffer A (50 mM Tris-HCl, 500 mM NaCl, pH 8) using HiTrap® immobilized metal affinity chromatography (IMAC) Fast Flow column charged with cobalt chloride. The protein can be eluted using a gradient of Buffer B (50 mM Tris-HCl, pH 8, 500 mM NaCl, 500 mM imidazole) with an increasing concentration of imidazole ranging from 10mM-500 mM. The desired fractions can be dialyzed using 50 mM Tris-HCl, 500 mM NaCl at pH 8.

The Q protein may be used to form hydrogels. The wound healing agents may be added before, during or after the formation of the hydrogels. For example, exosomes may be added during the formation of the hydrogels. The triterpenoids may be added once the gel is formed. For examples, the Q protein may first be allowed to form a gel (using buffered water) and the a solution comprising a one or more triterpenoids (e.g., CDDO-Im) is added atop the gel and the solution is allowed to imbibe through the gel for a period of time (e.g., over a period of days (e.g., 24-48 hours)). After the CDDO-Im has been imbibed into the gel, the solution from on top of the gel is removed.

In an embodiment, Q is complexed with exosomes to generate Exo-Q. Exosomes package the bioactive components of multipotent stromal cells, the cumulative effect being higher than administration of individual components alone. Exosomes suitable for the present compositions can be harvested with particular culture and passaging protocols, such as at 5% O₂ and 5% CO₂, 37° C. The O₂ may be varied from 4 to 8%, the CO₂ may be varied from 5-8% and the temperature may be varied from 34-40° C. It is preferable to culture the cells under hypoxic conditions, and therefore, preferably, the O₂ is less than 10%. In embodiments, the cells may be cultured at O₂ levels of 4, 5, 6, 7 and 8%. In embodiments, the cells may be cultured at O₂ levels of from 4 to 6%.

Multipotent stromal cells (such as from bone marrow) can be obtained and cultured. Bone marrow derived multipotent stromal cells (BM-MSCs) can be cultured as monolayer adherent cell cultures so that they undergo population doublings (such as 5-10 doublings). Cells are maintained in culture media containing serum (such as 15% fetal bovine serum). Upon reaching semi confluency, cells can be trypsinized and replated. Prior to harvest (such as 48 hours prior to harvest), the monolayer of bone marrow-derived multipotent stromal calls can be washed and culture media without exosomes is added, and the cell culture plates are returned to the humidified incubators with 5% O₂ and 5% CO_{2, 37}° C. Conditioned medium from these cell cultures is then harvested for the collection of exosomes. Size and density based separation techniques, such as differential centrifugation, density gradients, and size exclusion may be used to harvest and isolate exosomes. These techniques are known in the art. In general, initial low speed centrifugation is used to remove dead cells and cell debris. High speed centrifugation can then be used to collect exosomes in the pellet. The exosomes may then be suspended in suitable buffer. Preforming the steps at low temperature (such as 4° C.) minimizes alterations to proteins, lipids, and RNA content of the exosomes. The exosomes can then be purified by density gradient. Preferably, the exosome preparation exhibits tetraspanin protein and does not exhibit markers for apoptotic bodies or golgi. By using the process described herein, the exosome preparation displayed a mean diameter of about 170 nm±10 nm.

In an embodiment, this disclosure provides an enriched preparation of exosomes. Typically, it was found that the larger the number of cells, the larger the yield of exosomes. The initial preparation can be filtered through a 0.22 micron filter, so it effectively filters out any remnant larger extracellular vesicles (larger than 220 nm). The centrifugation and size exclusion steps ensure removal of microvesicles, apoptotic bodies and Golgi components. Per nanoparticle tracking analysis, the mode of exosome size is 150 nm, with sizes ranging from 30 nm (instrument lower limit of detection) to 250 nm. In an embodiment, the 90^{th} percentile for size distribution is 250 nm.

For the preparation of EXO-Q, exosomes can be added to the composition comprising Q prior to gelation. Exosomes may be in the range of 1×10⁶ to 1×10¹¹ per mL of the composition comprising Q. For example, 1×10⁹ to 5×10⁹ exosomes can be added into a mL of Q prior to gelation. In embodiments, 1×10⁹ to 15×10⁹ exosomes may be added per mL. The concentration of Q can vary from 1 mM to 5 mM. For example, the concentration of Q can be from 1 mM to 3.5 mM. The temperature range can vary from 4° C. to RT (under specific pH conditions). For example, Q may be in buffer solution (50 mM Tris-HCl, 500 mM NaCl, pH 8). The gels can be formed in the range of pH 7.4-10. In an embodiment, the other buffer conditions including PBS, pH 7.4, may be used. In an embodiment, about 1 to 3 billion exosomes are added in to 200 μL composition of 2 mM Q.

Q protein was originally designed to form robust nanofibers (purified under denaturing conditions), which can bind small hydrophobic molecules such as curcumin, to form meso-scaled fibers (Hume 2014, Biomacromolecules 2014 15 (10), 3503-3510). Gelation of Q at 4° C. (under native conditions) was a surprising observation. Addition of curcumin to Q protein, prior to gelation, adversely impacts the self-assembly and network formation. As such, it was surprising that when Q protein was mixed with exosomes, Q successfully formed a gel. Also surprisingly, addition of exosomes to Q protein did not impact the network formation. Instead, transmission electron microscopy (TEM) image of Exo-Q showed that exosomes were interspersed within Q fibrous network as further described in the example below.

A hydrogel may comprise a network of entangled fibers of Q protein (e.g., having SEQ ID Nos: 1 or 2 or a protein having at least 85% homology with SEQ ID Nos:1 or 2, or there may be some protein molecules that have SEQ ID NO:1 and some SEQ ID NO:2). In an example, the hydrogel does not comprise one or more crosslinked (e.g., does not comprise covalently crosslinked) Q protein fiber(s). In an embodiment, the hydrogel comprises physically crosslinked fibers. In an embodiment, the hydrogel comprises only physically crosslinked fibers and no chemically crosslinked fibers. In an embodiment, the hydrogel also comprises chemically cross-linked fibers. In an embodiment, the fibers non-covalently associate via one or more non-covalent interactions (e.g., hydrophobic interactions, π-π interactions, hydrogen bonds, and the like, and combinations thereof).

A hydrogel can comprise various amounts of water. In various examples, a hydrogel comprises 80 to 90%, such as 85 to 95% by weight (based on the total weight of the composition) water. In an embodiment, the hydrogel comprises about 91 to 95% by weight percent water. In an embodiment, the hydrogel comprises about 93 weight percent water. Addition of the wound healing agents has been shown to increase the storage modulus of Q, suggesting a higher crosslinking degree after wound healing agents are added. The increase may be around a 1.5 to 2-fold increase in storage modulus. In various embodiments, the concentration of Q in the hydrogel is 2 mM (1.3% w/v).

In an embodiment, this disclosure provides a hydrogel comprising fibers of Q protein (having SEQ ID Nos: 1 or 2 or a protein having at least 85% homology with SEQ ID Nos:1 or 2) and having distributed therein exosomes, wherein the exosomes may be derived from cultures of multipotent stromal cells. The exosomes may have a mean diameter of 160 to 180 nm. In an embodiment, at least 70%, 80%, 90%, 95%, 96%, 97%, 98% or 99% of the exosomes have diameter from 120 to 180 nm (e.g., 160 to 180 nm or 120 nm, 130 nm, 140 nm, 150 nm, 160 nm, 170 nm, or 180 nm). In an embodiment, 90% of the exosomes are less than 250 nm. The exosomes are not incorporated in a pore formed by the nanofibers, as was the case with small molecules associating with the nanofibers. Rather the exosomes are bound to the fibers and the addition of the exosomes during gelation does not affect fiber network formation. The exosomes may be associated with the Q protein fibers by non-covalent interactions. In an embodiment, about 1 billion to 3 billion exosomes may be present in 200 μl of the gel.

In an embodiment, instead of exosomes, triterpenoids, such as CDDO-Im, (1-(2-cyano-3,12-dioxooleana-1,9-dien-28-oyl) imidazole) are added to the Q hydrogel, where Q has is SEQ ID NO:1 or SEQ ID NO:2, or a peptide having 85% of SEQ ID NO:1 or SEQ ID NO:2 to generate Nrf2-Q. Addition of CDDO-Im was observed to increase storage modulus (G′), indicative of increased elasticity, a feature that can improve the ease of application to skin and open wounds/ulcers. Semi-synthetic triterpenoids, like CDDO-Im, are considered to be highly potent inducers of the Nrf2/Keap 1 pathway which regulates antioxidant and cytoprotective responses. CDDO-Im induces the nuclear translocation of the antioxidant response regulator Nrf2. Nrf2 binds to antioxidant response element (ARE) sequences in the promoter region of genes that improve defense against reactive oxygen species and maintain cell integrity. CDDO-Im may be added to the Q hydrogel during gelation.

In an embodiment, this disclosure provides a hydrogel comprising fibers of Q protein (having SEQ ID Nos: 1 or 2 or a peptide having at least 85% homology with SEQ ID Nos:1 or 2) and having distributed therein CDDO-Im. The CDDO-Im may reside within the pores formed by the Q fibers or a peptide variant thereof and/or be dispersed within the fibrous network of the hydrogel. Exosomes, on the other hand, are considered to be too large to reside within the pores and are present as dispersed throughout the fibrous network of the hydrogel.

In an embodiment, this disclosure provides a hydrogel comprising fibers of Q protein (having SEQ ID Nos: 1 or 2 or a peptide variant thereof having at least 85% homology with SEQ ID Nos:1 or 2) and having distributed in the hydrogel, exosomes and CDDO-Im. In embodiments, gels comprising from 1 μM to 10 mM triterpenoids (such as CDDO-Im) may be used. In embodiments, gels comprising 10 μM to 1 mM, 10 μM to 100 μM, 10 mM to 1 mM triterpenoids (such as CDDO-Im) and all values and ranges from 1 μM to 10 mM may be used. These values may correspond to the concentrations prior to gel formation.

For wound dressings, a gel like consistency may be used. It may be desirable that Q hydrogels exhibit an elastic modulus (storage modulus) in the range of 50 to 150 Pa.

The amount of exosomes used for a particular wound area may be adjusted based on the wound size and other characteristics. For example, in an embodiment, about 3 billion exosomes were used to treat a 4×4 mm wound. For example, in an embodiment, a 100 μL volume comprising about 3 billion exosomes may be used to treat a 10 mm in diameter wound (area 78.55 mm²) (e.g., full thickness/excisional wound) on a mouse spanning the depth of all layers of skin. A 100 μL volume is sufficient to occupy the gap in tissue in the skin.

An unexpected observation for the present compositions was the use of upper critical solution temperature (UCST) type gel for treatment of chronic wounds, which exhibit a much lower UCST than physiological temperature. Typically, hydrogels for wound healing applications have transition temperature at or around 37° C. Exo-Q and Nrf2-Q gels solubilize at wound temperature (estimated to be ˜30-31° C. (Gethin 2018, Wound Rep and Reg, 26: 251-256), providing a sustained delivery of exosomes or synthetic triterpenoids at the site of wounds without using painful injection. The resultant gels unexpectedly do not disrupt or introduce new physical barriers to the wound healing progression. Additionally, the exosomes from multipotent stromal cells do not induce an inflammatory response even across species, as demonstrated by application in mice in a preclinical model.

A hydrogel can have one or more desirable propert(ies). In various examples, a hydrogel exhibits one or more desirable mechanical propert(ies), one or more desirable rheological propert(ies), or a combination thereof. In an embodiment, a hydrogel has an upper critical solution temperature (UCST) of 30 or lower. In embodiments, UCST at pH 8 may be about 16° C. At higher pH, the UCST may be at RT (generally considered between 20 and 24° C., such as 22° C.). It is considered that small hydrophobic molecules stabilize the gel at higher temperature, providing a sustained wound healing effect.

In an embodiment, this disclosure provides wound healing compositions for topical, intradermal, or transdermal applications comprising a hydrogel having distributed therein one or more wound healing agents. The hydrogel comprises fibers of Q protein (having SEQ IDs:1 or 2 or a protein having at least 85% homology with SEQ ID Nos:1 or 2). The wound healing agents may be multipotent stromal cell derived exosomes, CDDO-Im, or both. The composition may comprise pharmaceutical excipients or ingredients.

The present compositions for topical or transdermal application can be produced in any solid, liquid or semi-solid formulation, including creams, emulsions, anhydrous compositions, aqueous dispersions, oils, foams, lotions, gels, ointments, sprays or aerosols or any other form suitable via the skin or mucosal surface. The formulations can be incorporated into support materials that can be applied to a wound surface, such as, for example, bandages, gauzes, clothing, diapers, dressings, adhesive or non-adhesive patches, and the like. The formulations can also be incorporated into cosmetic materials, such as foundations, lipsticks, moisturizers, creams, masks, and the like.

The hydrogel compositions may include pharmaceutically acceptable excipients, such as, buffering agents, antioxidants, preservatives, colorants, carriers, diluents, adjuvants, salts, and the like. Suitable water soluble buffering agents include carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system about 6, such as between about 7 to about 10 and more preferably about 7 to about 9. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. The excipients may be present in amounts of from 0.001 to about 5% by weight, such as, for example, 0.01 to about 2% by weight. Pharmaceutical composition ingredients and preparation can be carried out by standard pharmaceutical formulation techniques such as those disclosed in Remington: The Science and Practice of Pharmacy (2012) 22nd Edition, Philadelphia, PA. Lippincott Williams & Wilkins.

The topical, intradermal, or transdermal compositions may additionally comprise one or more of antibiotics, anti-septic agents, other wound healing agents, or pain relievers.

In an aspect, this disclosure provides a method for delivering a wound healing agent to a wound comprising contacting a hydrogel of the present disclosure to or near the wound. The hydrogel may be disposed on the wound. The hydrogel comprises fibers of Q protein (having SEQ IDs:1 or 2 or a variant protein thereof having at least 85% homology with SEQ ID Nos:1 or 2) and further comprises exosomes, one or more triterpenoids (such as CDDO-Im), or a combination thereof, distributed in the hydrogel. In animal studies, administration of the topical therapies into excisional wounds of type II diabetic mice, which typically demonstrate a severely delayed wound healing phenotype, significantly improved wound healing.

In an embodiment, the present disclosure provides compositions and methods for delivering wound healing agents to wounds, including chronic wounds, such as chronic ulcers. The wounds maybe associated with diabetes, burns, venous disease, pressure ulcers, age-associated complications, or any disorder that compromises the barrier function and integrity of skin. High oxidative burden in the tissues of patients with diabetes is at the pathogenic roots of many complications secondary to diabetes, including poor wound healing and amputation. Oxidative stress is also implicated in pathologic healing of chronic and age-related contexts. A temporary increase in reactive oxygen species is expected and necessary to physiologic healing of cutaneous wounds. In patients without diabetes, endogenous antioxidant defense pathways, like nuclear factor erythroid 2-related factor 2 (Nrf2), transcriptionally upregulate genes that regenerate reducing equivalents, directly neutralize reactive oxygen species, and restores levels of metabolic enzymes. This system is unable to neutralize elevated oxidative loads characteristic of hyperglycemia secondary to diabetes. By exogenously enhancing this pathway, oxidative stress is reduced in wounded tissue of diabetic patients and the delay in closure in diabetic wounds is also reduced.

In an aspect, this disclosure provides a method of treatment of wounds by applying to the wound or near the wound a hydrogel composition comprising fibers of Q protein (SEQ ID NO:1 or 2, or comprising some molecules of SEQ ID NO:1 and some SEQ ID NO:2), having distributed therein one or more wound healing therapeutic agents. In embodiments, the wound healing therapeutic agents may be exosomes (e.g., isolated from cultures of multipotent stromal cells, which may be human stromal cells) and/or triterpenoids. The wounds may be chronic wounds, such as diabetic wounds and chronic ulcers.

In an embodiment, this disclosure provides a therapeutic applicator for applying to or near a wound, said applicator comprising hydrogel comprising fibers of Q protein (SEQ ID NO:1 or 2, or comprising some molecules of SEQ ID NO:1 and some SEQ ID NO:2), having distributed therein one or more wound healing therapeutic agents. In embodiments, the wound healing therapeutic agents may be exosomes (e.g., isolated from cultures of multipotent stromal cells, which may be human stromal cells) and/or triterpenoids.

In the treatment of wounds, such as diabetic ulcers, following debridement of chronic diabetic ulcers, Exo-Q or Nrf2-Q can be applied topically for therapy. As described herein, both Exo-Q and Nrf2-Q have demonstrated efficacy with a single dose in mouse models, indicating a single or a few dosage (e.g., limited dosage) will be sufficient on human skin also. This dosage will circumvent needs for daily follow-up with trained clinical staff. As both hydrogels are biodegradable, treated wounds can be covered with standard wound dressings (i.e. gauze, non-adherent dressings) dictated by physician preference. In addition to diabetes associated wounds, a wide range of chronic wounds, including but not limited to, pressure ulcers, venous insufficiency/disease, age, surgery, or autoimmunity can be treated by the present compositions and methods. Exo-Q and Nrf2-Q may be combined with skin bilayers, cellular/acellular scaffolds, bio-ink printed skin constructs/scaffolds for coverage of large exposed areas of skin.

In additional embodiments, exosomes and triterpenoids (e.g., synthetic triterpenoids) may be combined in a single or layered Q based hydrogel. Exosomes are lipid bilayers that naturally transport lipids and membrane bound molecules between cells. In hydrogels where both exosomes and synthetic triterpenoids are used, the triterpenoids may be incorporated into the exosomes and distributed in the hydrogel.

Advantages of the present compositions and methods include the following. Since 1997, not a single drug has been approved by the FDA for efficacy in treatment of chronic wounds, including diabetic ulcers. The present disclosure can provide therapeutic intervention for chronic wounds, not just another modality for palliative wound care. A single dose of Exo-Q or Nrf2-Q has demonstrated highly significant acceleration of wound closure in preclinical type II diabetic mouse models, compared to untreated wounds. Exo-Q or Nrf2-Q are easy to use and apply in the form of a topical application, allowing for administration by a wider range of care providers, even patients themselves. This reduces need for highest clinical training and allows better allocation of skill and time of all clinical staff. Current care approaches require daily dressing changes in clinic/hospital settings. The limited dosage of Exo-Q and Nrf2-Q, applied during acute phases following debridement by a clinician, reduces chances of patient non-compliance during the care period. Exo-Q gels melt at lower temperatures, providing a sustained release of exosomes at human physiological temperatures on the skin surface. This feature ensures that maximum efficacy of Exo-Q is only active following contact with human skin. Exo-Q and Nrf2-Q are biodegradable products that do not interfere with the wound healing progression. Exo-Q delivers a cell-derived therapy, and thereby removes the apprehensions surrounding use of multipotent stromal cells for wound healing. It also does not appear to induce inflammatory response, even across species. Nrf2-Q treats the underlying pathology of delayed wound healing by augmenting endogenous defense against reactive oxygen species. Unexpectedly, it also enhances endogenous growth factor signaling. Nrf2-Q demonstrates activation followed by inhibition of the Nrf2 pathway, which may prevent prolonged activation and off-target side effects.

The following Statements are various embodiments and examples of the present disclosure.
Statement 1. A composition for wound healing comprising a hydrogel having distributed therein one or more wound healing agents, wherein the hydrogel comprises nanofibers comprising a protein having a sequence of SEQ ID NO. 1.
Statement 2. A composition of Statement 1, wherein the wound healing agents are exosomes produced by human multipotent stromal cells.
Statement 3. A composition of Statement 2, wherein the exosomes are obtained from the conditioned medium of a culture of multipotent stromal cell.
Statement 4. A composition of Statement 3, wherein the multipotent stromal cells are obtained from bone marrow.
Statement 5. A composition of any one of the preceding Statements, wherein the hydrogel comprises 85 to 95 weight percent of water, and/or the nanofibers (e.g., Q fibers) are present at a density of 1 mM to 5 mM, and/or the concentration of exosomes is 1×10⁶ to 1×10¹¹ per mL.
Statement 6. A composition of any one of the preceding Statements, wherein the concentration of exosomes is 5×10⁹ to 15×10⁹ per mL of a 2 mM hydrogel comprising nanofibers (e.g., Q composition).
Statement 7. A composition of claim 1, wherein the wound healing agent is a triterpenoid.
Statement 8. A composition of claim 7, wherein the triterpenoid is CDDO-Im.
Statement 9. A composition of Statements 7 or 8, wherein the hydrogel comprises 85 to 95 weight percent, and/or the nanofibers (e.g., Q fibers) are present at a density of 1 mM to 5 mM, and/or the concentration of triterpenoids in the hydrogel is 1 μM to 10 mM.
Statement 10. A composition of Statement 9, wherein the concentration of triterpenoids in the hydrogel is 10 μM to 100 μM.
Statement 11. A composition of any one of Statements 7-10, wherein the triterpenoid is CDDO-Im.
Statement 12. A method of treating a wound comprising applying to the wound a composition of any of the preceding Statements.
Statement 13. A method of Statement 12, wherein the composition is incorporated in or disposed on a material that can be applied to the wound.

The following examples are provided as illustrative examples and are not intended to be restrictive in any way.

### Example 1

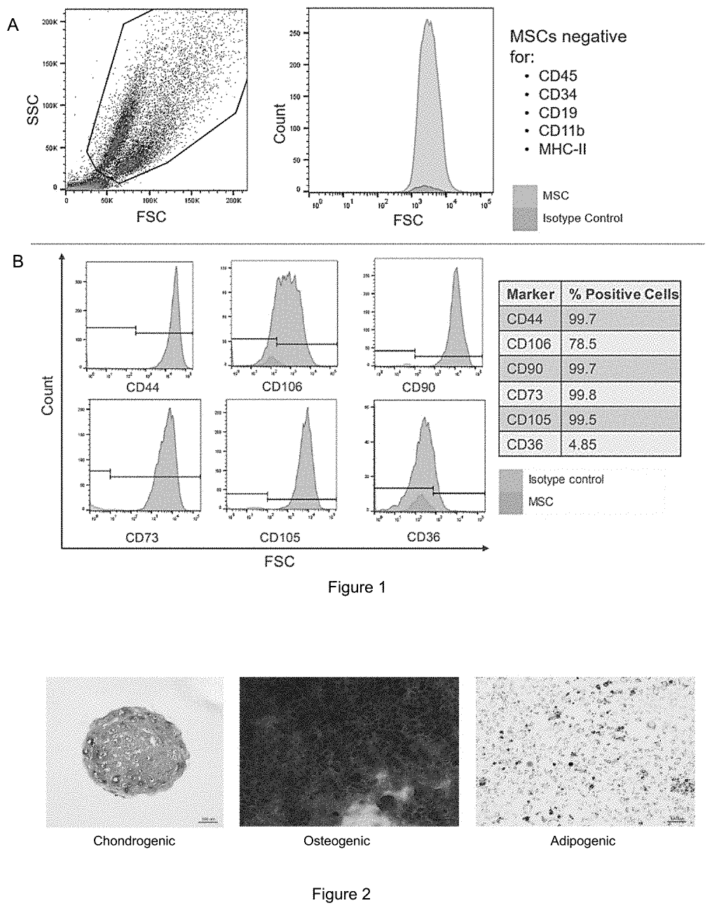

Bone marrow was obtained from human donors by routing methods. We used healthy human bone marrow from donors aged 18-30, and seeded cells onto tissue culture-treated petri dishes. 1×10⁶ cells were allowed to undergo population doublings. We used bone marrow-derived multipotent stromal cells (BM-MSCs) that have undergone 8-9 population doublings, from the time of bone marrow aspiration and being plated in monolayer adherent cell culture. Cells were maintained in alpha-MEM supplemented with 15% fetal bovine serum (FBS), 1% non-essential amino acids, and 1% penicillin/streptomycin, at 37° C./5% CO_{2/5}% O₂. Attempts with Dulbecco's MEM (DMEM) failed to sustain healthy cells, similar to the more traditional 10% FBS for adherent cell culture. When the cells were about 80% confluent in a 150 mm plate (−15-18×10⁶ cells), cells were trypsinized to detach them from the plastic plate and cells were passaged 1:3. A representative characterization of the BM-MSCs is shown in FIG. 1. When cells were passaged prior to or beyond the 85% confluency point, reduced yield of exosomes and greater differentiation of the cells was observed. As such, generally a confluency of about 75 to 80% is desirable.

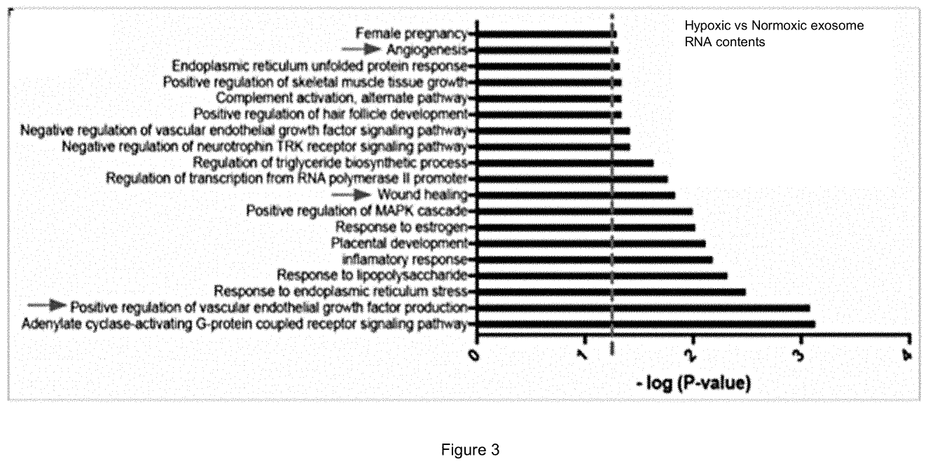

The hypoxic conditions we used in our culture of BM-MSCs, mimic the bone marrow's native hypoxic environment. We have previously observed that hypoxic conditions also favor retention of primitive cell properties and enable expansion in cell culture. Maintenance of the cells in hypoxia prolongs their plasticity or ability to differentiate along multiple mesenchymal lineages (FIG. 2). On the other hand, culture in normoxia (atmospheric oxygen levels) promoted differentiation of the BM-MSCs, limiting their proliferation capacity and prevented us from expanding the cells to sufficient passages/quantities for exosome collection. Further, we have confirmed that the hypoxic (5% O₂) conditions we use in our cell culture protocol optimally prime the exosomes for wound healing functions when compared to exosomes generated from BM-MSCs in normoxia or atmospheric oxygen tension, all other cell culture conditions were identical. High throughput sequencing of RNA, facilitated by microfluidics in a flow cell, packaged in and carried by exosomes shows significant enrichment for RNAs that regulate molecular pathways known to be critical for wound healing processes (FIG. 3).

### Example 2

This example describes isolation of exosomes. At passage 3 (after the first 8-9 population doublings) 70-80% confluent adherent BM-MSCs are washed twice with 1×PBS without Ca²⁺/Mg²⁺. Cells are then incubated for 48 hours at 37° C./5%CO2/5%O₂ in alpha-MEM containing nucleosides, supplemented with 15% exosome-depleted fetal bovine serum (FBS), 1% non-essential amino acids, and 1% penicillin/streptomycin. The conditioned media was then pooled and exosomes were harvested from conditioned media by differential ultracentrifugation. Briefly, conditioned media was centrifuged at 300×g for 5 minutes to remove dead cells and cell debris, 2,000×g for 10 minutes to remove apoptotic bodies, 10,000×g for 30 minutes to remove microvesicles, and then 100,000 g×90 minutes to pellet exosomes. Inclusion of microvesicles or apoptotic bodies alters the size range of our isolated extracellular vesicles, and interferes with isolating a uniform type of vesicle, and subsequently confounds characterization of the vesicles. Exosome pellets were resuspended in 1×PBS w/Ca²⁺Mg²⁺, pooled into a single tube and centrifuged for 100,000 g×90 minutes to re-pellet total harvest. Exosome pellets were resuspended in 200 μL of 1×PBS w/Ca²⁺Mg²⁺. All steps were performed at 4° C. to minimize changes in the proteins, lipids and RNA content being carried by exosomes. The exosome isolate was then purified to remove other non-specific free proteins through a density gradient. The exosomes are then purified with qEV single 70 nm (Izon) size exclusion chromatography filters per the manufacturer's instructions. The first 1 mL of sample to pass was discarded and only the next 400 μL was used for analysis. Without the further purification steps, the isolate carries extracellular matrix-associated proteins and once again introduces variability for downstream use in wound healing treatment. To concentrate the sample, the collected volume is centrifuged at 100,000×g for 90 minutes at 4° C. and resuspended in 200 μL of 1×PBS w/Ca²⁺Mg²⁺ once more.

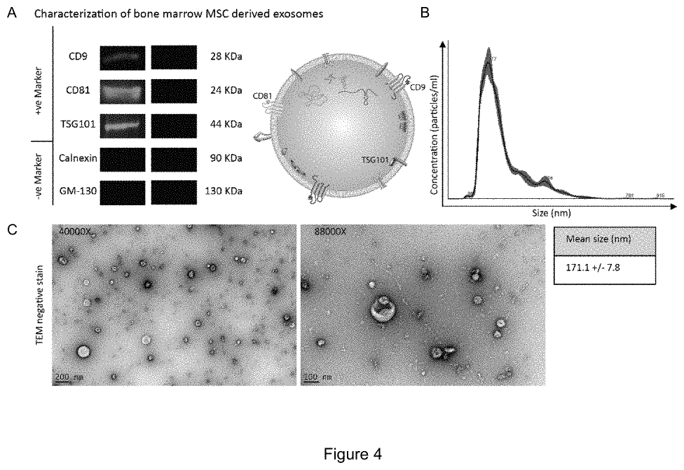

Our isolated exosomes, isolated by density gradient and size-exclusion chromatography display expected tetraspanin proteins and do not have markers for apoptotic bodies or Golgi (FIG. 4A). Using nanoparticle tracking analysis, we determined that our isolates have a mean diameter of 171.1±7.8 nm, in agreement with exosome size in published peer-reviewed literature (FIG. 4B). We imaged the exosome using transmission electron microscopy to further confirm their presence and size (FIG. 4C).

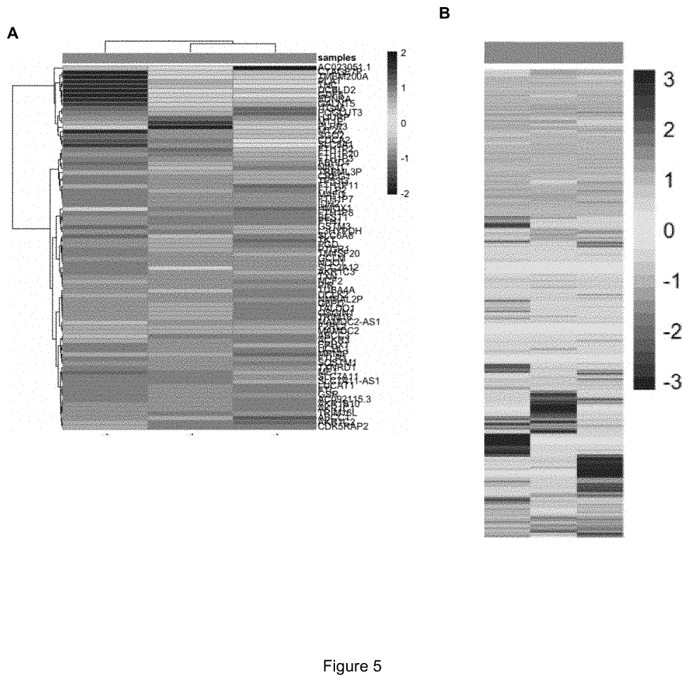

To characterize the total RNA content being carried by MSC exosomes, we performed next generation sequencing (performed by System BioSciences using their customized library prep) following the isolation methods described above. FIG. 5 demonstrates that we found very minimal variation among exosomal RNA content among different human donors. This analysis demonstrates that our optimized cell culture protocol ensures similar cell behavior and reliable exosome generation. FIG. 5 demonstrates that we found very minimal variation among MSC RNA indicated by the high degree of alignment of gene expression, even though the cells are from different human donors. The intensity heat map, showing range of expression of genes from high to low confirms that our optimized cell culture protocol ensures similar cell behavior response of MSCs. Standardization of the MSCs results in reliable exosome generation, and similar alignment of the exosomal RNA contents, as shown in the intensity heat map in FIG. 5B.

### Example 3

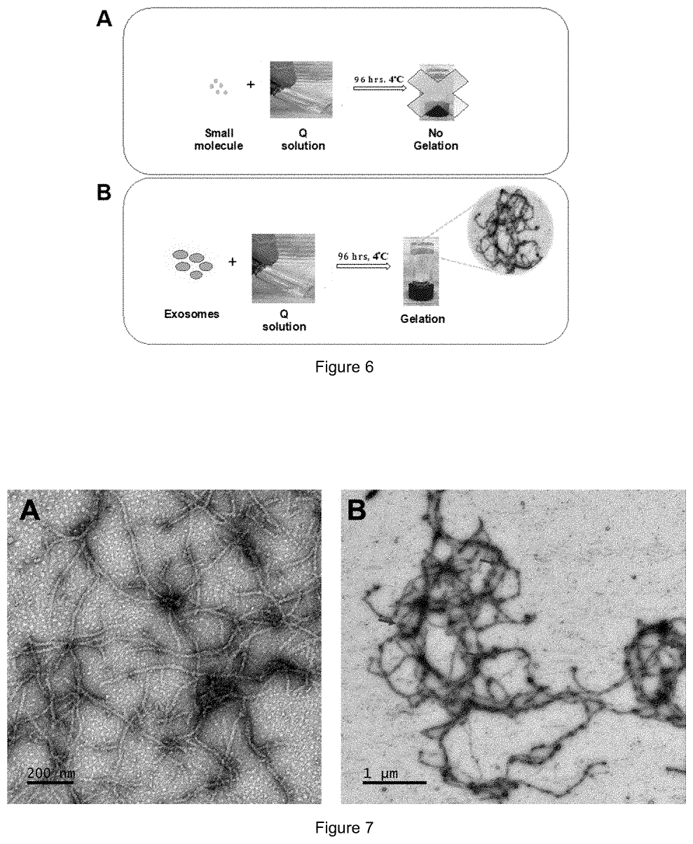

This example describes the preparation of hydrogel comprising exosomes. The addition of small molecules prior to gelation hinders network formation (FIG. 6a). In order for the drug to bind the Q gel, we use a stock solution of a small molecule and add it atop the gel. The drug molecules slowly imbibe through the gel and the excess drug is then later removed from the gel.

Given the larger size of the exosomes, the same approach of adding the exosomes after gelation would not result in its uniform distribution. In an unexpected observation, when a solution of Q protein was mixed with the exosomes, Q successfully formed a gel within the same time period as Q gel by itself (FIG. 6b). Interestingly, the exosomes did not impact the network formation and gelation. The exosomes are uniformly distributed throughout the gel and are interspersed with the Q fibers as observed by transmission electron microscopy (FIGS. 7A and B).

### Example 4

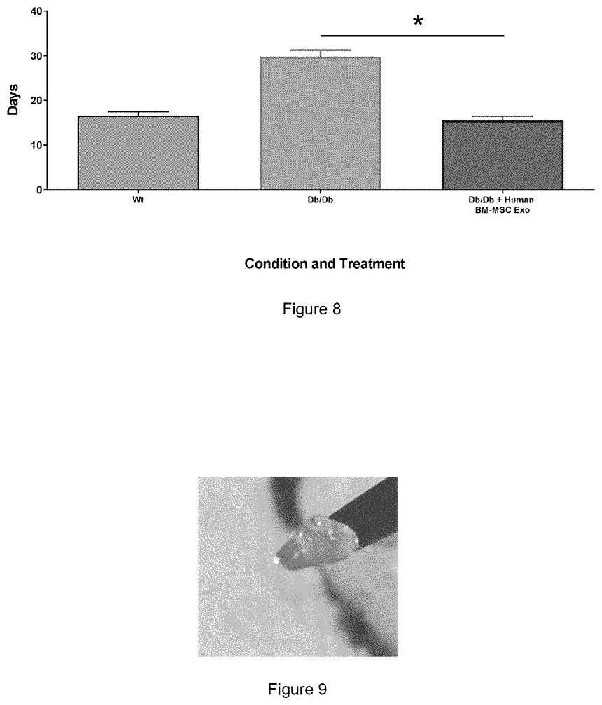

This example describes the use of the Exo-Q hydrogel in wound healing. 1×10⁹ exosomes (suspended in phosphate buffered saline containing 0.9 mM CaCl₂ and 0.49 mM MgCl₂-6H₂O) were administered through intradermal injections to the periphery of excisional wounds on a type II diabetic mouse model. This model displays wound healing delays to mimic the chronic nature of human diabetic wounds (Galiano 2004, Wound Repair Regen. 2004 Jul-Aug;12(4):485-92). The results were remarkable when compared to diabetic treated with just normal saline (FIG. 8). Critically important, the single and local exosome dose reduced time to closure of the diabetic wounds within range of wild type non-diabetic mice. Though our results were promising, the administration method was not practical or translatable. For patients to be able to self-administer the exosomes, a suitable delivery vehicle that would not diminish the therapeutic effect of the exosomes was needed.

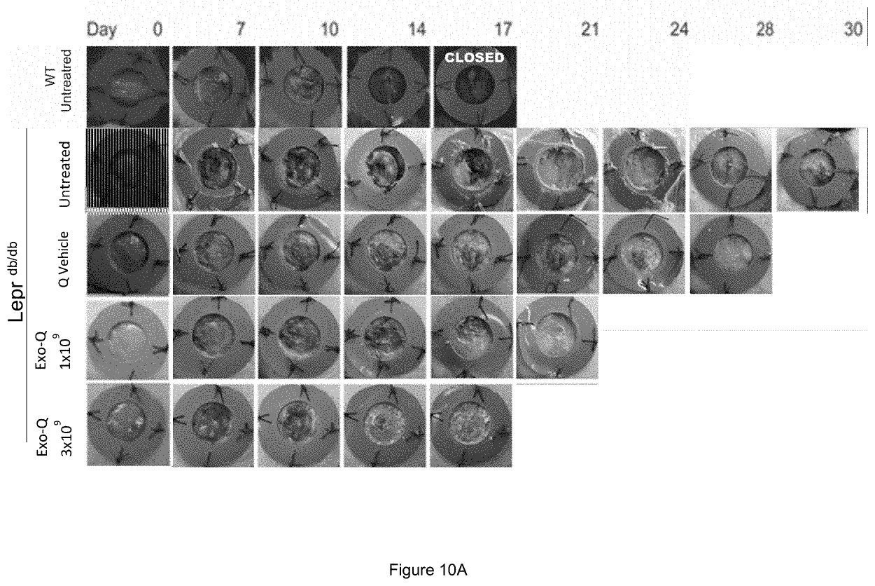

Next we tested delivering exosomes using the Q hydrogel. We had previously observed that addition of curcumin to Q protein (in 50 mM tris hydrochloride, pH 8, and 500 mM NaCl), prior to gelation, adversely impacts the self-assembly and network formation. In an unexpected observation, when Q protein was mixed with exosomes, Q successfully formed a gel. Surprisingly, addition of exosomes to Q protein did not impact the network formation. Instead, transmission electron microscopy (TEM) image of Exo-Q (diluted to 50 uM) shows that exosomes were interspersed within Q fibrous network (FIG. 7B). To establish the pre-clinical therapeutic efficacy, we used the well-established type II diabetes mouse model that exhibits severe delays in wound healing, when compared to wild type counterparts. This is the same model we used in our preliminary studies described in the previous section. FIG. 9 demonstrates the Exo-Q immediately prior to topical application on the mouse diabetic wounds. When we administered Exo-Q to 6 mm diameter diabetic wounds 1 day post-excision, we found highly significant acceleration of time to closure from 20 days to 15 days (not shown). A single, local and topical application of Exo-Q was sufficient to achieve these significant healing outcomes.

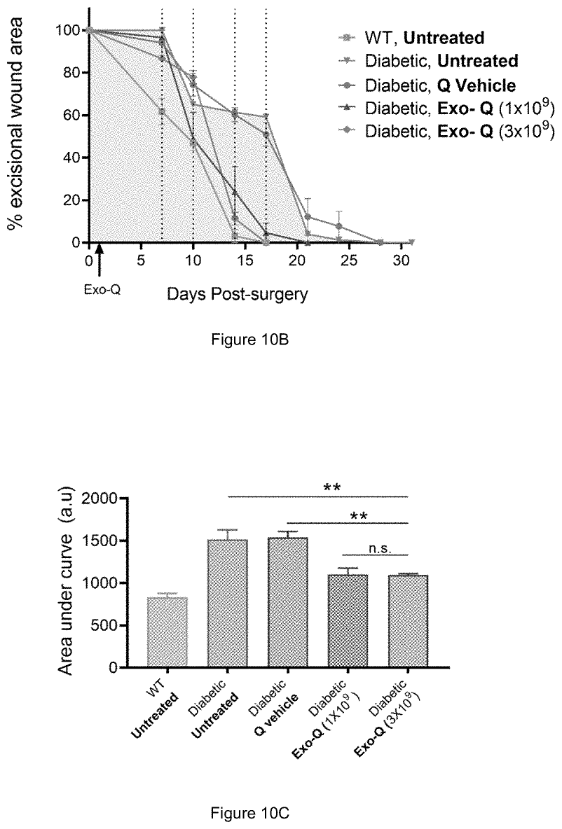

We administered Exo-Q to 10 mm diabetic wounds on mice 1 day post-excision and found a highly significant dose-dependent acceleration of wound closure to 21 days and 17 days following single 1 billion and 3 billion exosome doses in Exo-Q, respectively, compared to the 30 days required by untreated diabetic wounds (FIG. 10A). Plotting the wound area over time revealed that at day 7 post-wounding and treatment, the wound areas are significantly different by ANOVA, with the WT wounds being significantly different from all the diabetic ones (FIG. 10B). By day 14 and 17, the wound areas are once again significantly different by ANOVA, but this time the WT and Exo-Q (both doses) treated diabetic wounds are significantly different from the untreated and Q-vehicle treated diabetic wounds (n=6 for each treatment). Integration of the area under these curves (AUC, in arbitrary units, a.u), a prognostic measure indicative of wound closure potential, found lower AUC of Exo-Q-treated wounds in contrast to untreated and Q vehicle-treated diabetic wounds (FIG. 10C). This result confirms that the Exo-Q treated diabetic wounds have significantly higher healing potential compared to the untreated and Q vehicle-treated wounds, similar to WT wounds with physiologic healing. The Exo-Q treatment of diabetic wounds alters their wound healing trajectory within range of WT acute healing wounds.

### Example 5

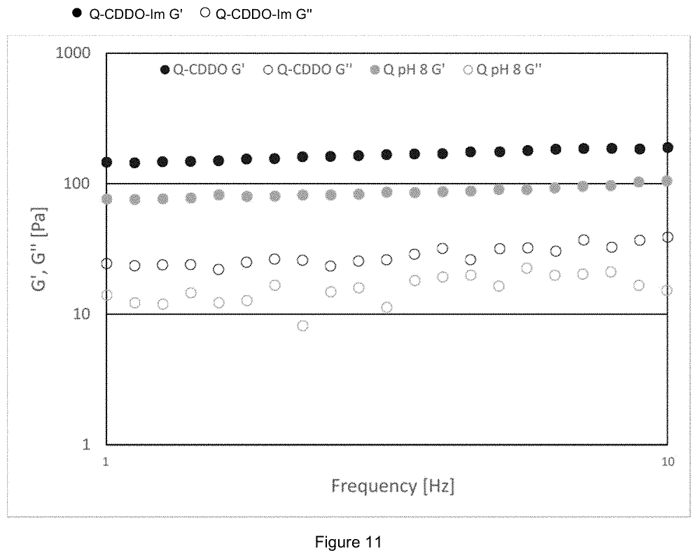

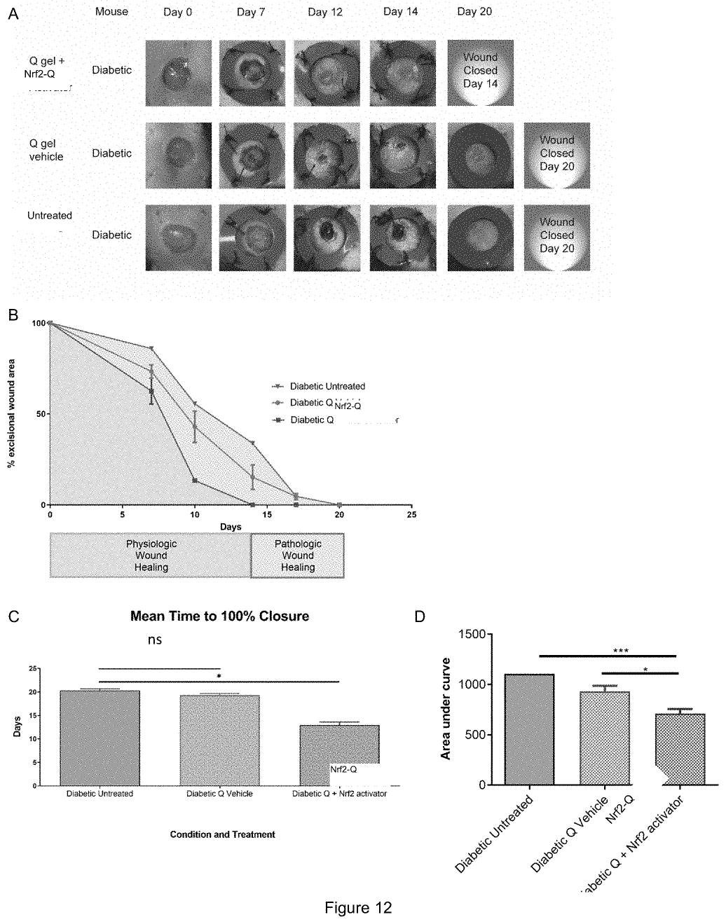

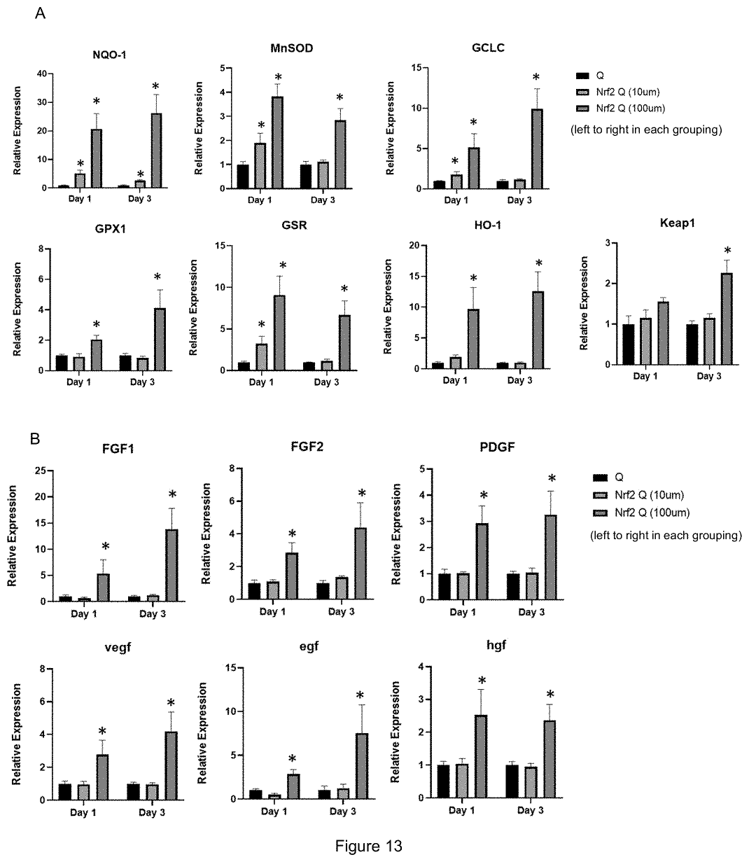

This examples describes the use of Q hydrogel wherein Q is complexed with CDDO-Im (with 1-(2-cyano-3,12-dioxooleana-1,9-dien-28-oyl) imidazole to generate Nrf2 Q. Addition of CDDO-Im exhibited an increase in storage modulus (G′) (FIG. 11), indicating increased elasticity, a feature that will improve the ease of application to skin and open wounds/ulcers. We topically applied Nrf2-Q in our mouse model of delayed type II diabetic wound healing with wounds 6 mm in diameter. Compared to vehicle (Q alone), a single application of Nrf2-Q significantly reduced time to closure from 20 days to 14 days (FIG. 12, n=3). Analysis of gene transcripts of Nrf2-Q-treated wounds demonstrates major increases in many cytoprotective antioxidant gene targets of Nrf2, such as NQO-1, MnSOD, GCLC, GPX1, GSR, and HO-1 at both 10um and 100um concentrations of Nrf2-Q gel (FIG. 13A). Interestingly, activation of Nrf2 also increases transcription of its own inhibitor, Keap1. This negative feedback loop allows the wound tissue to limit excessive Nrf2 activity following administration of Nrf2-Q and re-introduce homeostatic redox regulation (Lee 2007). Surprisingly, Nrf2-Q also results in transcriptional upregulation of a number of growth factors critical for wound healing, such as FGF1, FGF2, PDGF, VEGF, EGF, and HGF (FIG. 13B). Growth factors have long been considered for therapeutic applications including wound healing modalities, but their biodegradability, inactivation, and interaction with unique biochemistry and matrix of the wound microenvironment have been barriers to translation. Nrf2-Q presents an opportune and unique tool to induce a broad range of beneficial and effective growth factors by targeting a master regulatory pathway.

Example 6

This examples describes wound histology, gene expression analysis of Exo-Q treated diabetic wounds, and the thermoresponsiveness of Exo-Q hydrogels.

### Wound Histology.

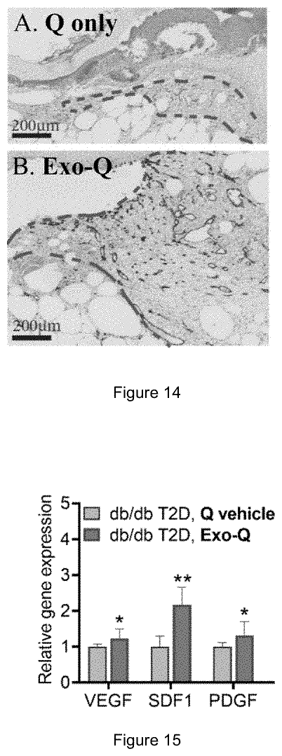

At 10 days post-wounding and treatment with Exo-Q (3×10⁹ dose), we analyzed the wound architecture of diabetic wounds by harvesting wound and immediately peripheral skin tissue and cross-linking in 4% paraformaldehyde, and processing into paraffin blocks and sectioning at 5 μm. Immunostaining wound tissue sections with antibody to CD31, which detects endothelial cells, coupled with hematoxylin staining for nuclei, showed that Exo-Q treated diabetic wounds have extensive granulation tissue area (indicated by dashed line) with extensive neovascularization (brown CD31+staining) in sharp contrast to Q vehicle-treated ones (FIG. 14). Vascularized granulation tissue is a yardstick of wound healing progression.

### Gene Expression Analysis of Exo-Q Treated Diabetic Wounds.

Additionally, we performed qPCR of critical wound healing associated genes at this same time point using the wound tissues. We collected 20 mm diameter total tissue around the initial 10 mm diameter wound, homogenized and lysed the whole tissue using ceramic beads in Trizol (ThermoFisher). Following chloroform phase separation and ethanol-based RNA precipitation, we isolated RNA using a Qiagen RNeasy kit. We performed first strand synthesis using High Capacity cDNA Reverse Transcription kit using 500 ng RNA. We found significant changes in gene expression of VEGF, SDF1 and PDGF in Exo-Q treated diabetic wounds in comparison with Q vehicle-treated ones (FIG. 15).

### Thermoresponsiveness of Exo-Q Hydrogels.

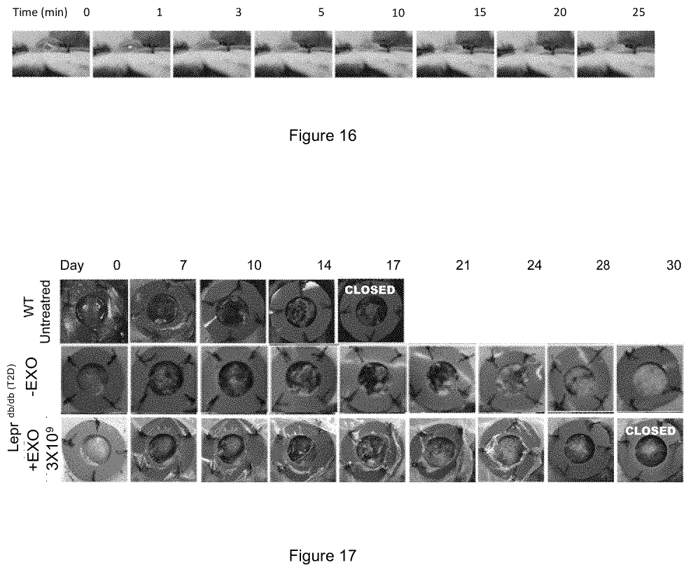

The Exo-Q hydrogels are thermoresponsive, as Q forms UCST hydrogels. To confirm our expectations, we applied Exo-Q hydrogels to wounds on the dorsum of mice and photographed the hydrogel from a fixed height at regular intervals to observe any changes in the consistency (FIG. 16). As the figure shows, we found that the viscous Exo-Q hydrogel (stored at 4° C.) undergoes a phase shift once in contact with the mouse skin at 31-32° C. within the first 30 minutes after application, transitioning from a domed hydrogel to a more fluid and flatter version. This was beyond any expectation for the phase change, as we anticipated a time frame in the order of hours. This feature enables contact of the Exo-Q hydrogel with the exposed cells of the diabetic wound bed.

While the present invention has been described through illustrative embodiments, routine modification will be apparent to those skilled in the art and such modifications are intended to be within the scope of this disclosure.

1. A composition for wound healing comprising a hydrogel having distributed therein one or more wound healing agents, wherein the hydrogel comprises nanofibers comprising a protein having a sequence of SEQ ID NO:1, SEQ ID NO:2, a protein having 85% homology of SEQ ID NO:1 or SEQ ID NO:2, or a combination thereof.

2. The composition of claim 1, wherein the wound healing agent is exosomes produced by human multipotent stromal cells.

3. The composition of claim 2, wherein the exosomes are obtained from the conditioned medium of a culture of multipotent stromal cell.

4. The composition of claim 3, wherein the multipotent stromal cells are obtained from bone marrow.

5. The composition of claim 1, wherein the hydrogel comprises 85 to 95 weight percent of water.

6. The composition of claim 1, wherein nanofibers have a concentration of 1 mM to 5 mM. The composition of claim 6, wherein the nanofiber concentration is 2 mM.

8. The composition of claim 7, wherein the protein has the sequence of SEQ ID NO:1.

9. The composition of claim 7, wherein the protein has the sequence of SEQ ID NO:2.

10. The composition of claim 1, wherein the concentration of exosomes is 1×10⁶ to 1×10¹¹ per ml of the hydrogel.

11. The composition of claim 10, wherein the concentration of exosomes is 5×10⁹ to 15×10⁹ per mL of the hydrogel.

12. The composition of claim 1, wherein the wound healing agent is triterpenoid.

13. The composition of claim 12, wherein the triterpenoid is CDDO-Im.

14. The composition of claim 12, wherein the hydrogel comprises 85 to 95 weight percent water.

15. The composition of claim 12, wherein the nanofibers have a concentration of 1 mM to 5 mM.

16. The composition of claim 12, wherein the concentration of triterpenoids in the hydrogel is 1 μM to 10 mM.

17. The composition of claim 16, wherein the concentration of triterpenoids in the hydrogel is 10 μM to 100 μM.

18. A method of treating a wound comprising applying to the wound a composition of claim 1.

19. The method of claim 18, wherein the composition is incorporated in or disposed on a material that can be applied to the wound.

20. The method of claim 18, wherein the wound is a wound associated with diabetes, burns, venous disease, pressure ulcers, or age-associated complications.
